(19) United States
(12) Patent Application Publication
(10) Pub. No.: US 2008/0300632 A1
(43) Pub. Date: Dec. 4, 2008

(54) Method of fabricating medical devices and medical devices made thereby

(76) Inventors: Michael S. Butler, St. Charles, IL (US); Kara A. Bucci, Palos Park, IL (US)

Correspondence Address: BOWMAN & ASSOCIATES, 1016 3rd Avenue, SW, Suite #106, CARMEL, IN, 46032, US

(21) Appl. No.: 12/156,354

(22) Filed: May 30, 2008

Related U.S. Application Data
(60) Provisional application No. 60/932,246, filed on May 30, 2007.

(51) Int. Cl. A61B 17/70; B21D 31/00

(52) U.S. Cl. 606/246; 606/280; 72/379.2

(57) ABSTRACT

A method of fabricating medical devices and medical devices made from the method of fabricating a medical device, the medical device, without being exhaustive, may be a medical instrument, an implant, a prosthetic, a body support structure or the like. The method includes work-hardening a work-hardenable metal to achieve a desired microstructure of the metal, then fabricating a medical device in accordance with the desired microstructure utilizing the work-hardened metal. In one form, the desired microstructure is an elongated grain structure. The medical device is created from the grain-elongated metal such that the medical device is oriented relative to the plane of grain elongation. In a particular form of the invention, the medical device is a curved spine plate wherein elongated grains of the work hardened metal are oriented in a plane normal to a curvature of the spine plate. Work-hardening includes forging, cold rolling or hot rolling, and annealing prior to use in medical device fabrication. This creates a metal implant stock that has more strength and flexibility in compression and bending than without undergoing the present work-hardening. These properties are exploited in medical device design, fabrication, fabrication orientation and/or the like to create medical devices such as super strong implants.

### RELATED APPLICATIONS

This patent application claims the benefit of and/or priority to U.S. Provisional Patent Application Ser. No. 60/932,246 filed May 30, 2007, entitled “Method of Fabricating Medical Device and Medical Devices Made Thereby” the entire contents of which is specifically incorporated herein by reference.

### BACKGROUND OF THE INVENTION

1. Field of the Invention

The present invention relates to method for fabricating medical devices such as instruments, implants, prostheses, body support structures and the like, and the instruments, implants, prostheses, body support structures and the like that are made thereby.

2. Background Information

The principles and processes of metalworking have been known for many years and are still developing. These principles and processes have allowed metalworkers to produce metals and/or metal stock that exhibit different properties. These properties can be exploited to provide desired characteristics from which to fabricate items. Metalworking processes such as forging, hardening, annealing and the like may be employed to create a metal or metal item whose properties or characteristics are determined by the process or processes used on the metal or metal item. The various metalworking processes can cause change in and to the metal or metal item.

It is known in some instances to take advantage of such changes in the fabrication of various metal items. For example, various types of steel may be produced and/or worked to make items such as cutlery, utensils, structural beams, tools, and the like. The different changes to the metal wrought by such metalworking processes makes different metal whose properties are exploited for a particular item. However, it has heretofore not been known to utilize metal engineered by metalworking to fabricate medical devices such as instruments, implants, body support structures and the like.

Accordingly, it is desirable to utilize one or more metal working processes to engineer structural properties of a metal stock and/or utilize that metal stock in the fabrication of medical devices such as instruments, implants, prostheses, body support structures and the like.

### SUMMARY OF THE INVENTION

In one form, the present invention provides a method of fabricating a medical device. In another form, the present invention is a medical device made from the present method of fabricating a medical device. Without being exhaustive, a medical device made in accordance with the present method may be a medical instrument, an implant, a prosthetic, a body support structure or the like.

A method according to the present principles includes work-hardening a work-hardenable metal to achieve a desired microstructure of the metal, then utilizing the work-hardened metal to fabricate a medical device in accordance with the desired microstructure.

In one form, the desired microstructure is an elongated grain structure. The medical device is created from the grain-elongated metal such that the medical device is oriented relative to the plane of grain elongation.

The medical device may be a spine implant such as a spine plate. In a particular form of the invention, there is provided a medical device comprising a curved spine plate made from a work hardened metal wherein elongated grains of the work hardened metal are oriented in a plane normal to a curvature of the spine plate.

Work-hardening includes forging, cold rolling or hot rolling, and annealing prior to use in medical device fabrication. Work-hardening may include one or more of forging, cold rolling or hot rolling, and annealing, or a series of one or more of forging, cold rolling or hot rolling, and annealing. This creates a metal implant stock that has more strength and flexibility in compression and bending than without undergoing the present work-hardening. These properties are exploited in medical device design, fabrication, fabrication orientation and/or the like to create medical devices such as super strong implants.

The medical devices fabricated from the present method include instruments, implants, prostheses, body support structures and the like. In one particular form and as described herein as a representative medical device and/or implant, are anterior cervical plates that may be part of an anterior cervical plate system (ACPS).

### BRIEF DESCRIPTION OF THE DRAWINGS

The above mentioned and other features, advantages and objects of this invention, and the manner of attaining them, will become apparent and the invention itself will be better understood by reference to the following description of embodiments of the invention taken in conjunction with the accompanying drawings, wherein:

FIG. 1 is a perspective view of a typical configuration of a medical device and particularly an implant configured as a one-level (1-L) static spine plate fabricated in accordance with the present principles;

FIG. 2 is a perspective view of a typical configuration of a medial device and particularly and implant configured as a two-level (2-L) dynamic spine plate fabricated in accordance with the present principles;

FIG. 3 is a sketch of a micrograph showing an X-Z cross section of a hardened metal in accordance with the present principles that is used for the fabrication of a medical device in accordance with the present principles, and particularly showing the desired microstructure property of grain elongation in the x-direction;

FIG. 4 is a sketch of a micrograph showing a Y-Z cross section of the hardened metal shown in FIG. 3, with equlaxed grains (no elongation in the y-direction);

FIG. 5 is a perspective view of an exemplary embodiment of a 1-L static spine plate fashioned in accordance with the present principles; and

FIG. 6 is a sectional view of the spine plate of FIG. 5 taken along line 6-6 thereof, particularly showing in an enlarged portion of the spine plate, the homogenous grain structure with elongated alpha grains (L to R direction) in accordance with the present principles.

Like reference numerals indicate the same or similar parts throughout the several figures.

A full description of the features, functions and/or configuration of the components depicted in the various figures will now be presented. It should be appreciated that not all of the features of the components of the figures are necessarily described. Some of these non discussed features as well as discussed features are inherent from the figures. Other non discussed features may be inherent in component geometry and/or configuration.

### DETAILED DESCRIPTION OF EMBODIMENTS OF THE INVENTION

It should be appreciated that the present metal fabrication method of utilizing one or more metal working processes to engineer structural properties of a metal stock and/or utilize that metal stock in the fabrication of a medical device includes all medical devices such as, without being exhaustive, instruments, prostheses, implants, body support structures and the like. While the present invention is being described in connection with spine plates, it should be appreciated that the spine plates are representative of all types of medical devices. Moreover, the microstructure property of grain elongation is an example and thus is not exhaustive of the microstructure properties that can be manipulated in accordance with the present principles and exploited through medical device design regarding the particular manipulated microstructure.

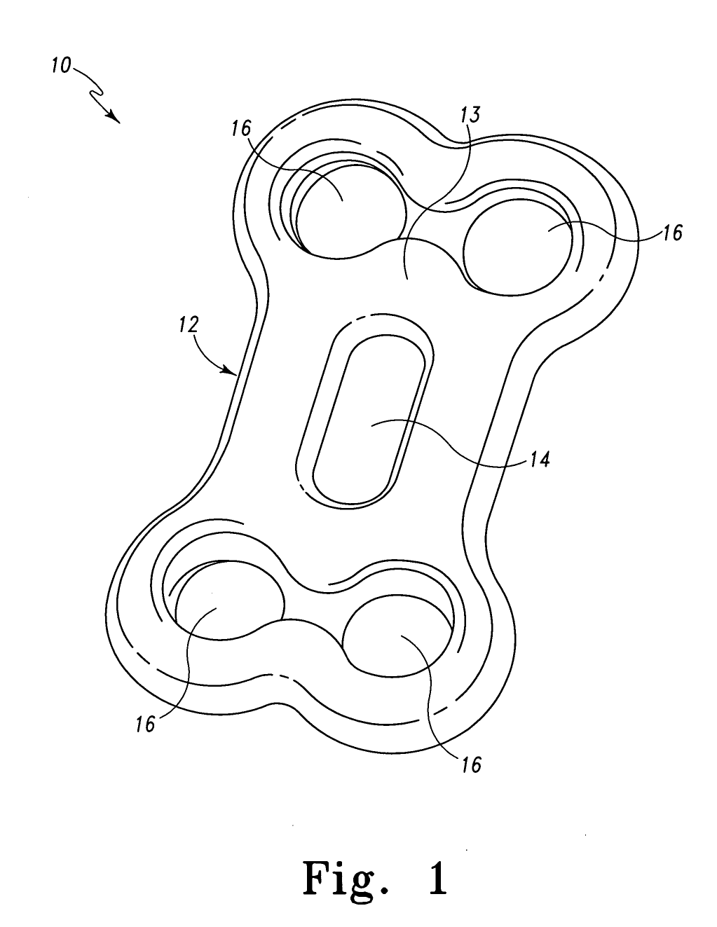

FIG. 1 shows a typical configuration of a medical device, particularly being a one-level (1-L) static spine plate generally designated 10 of which provides an example of a configuration of a spinal implant such as may be fabricated from a work hardened metal in accordance with a method according to an aspect of the principles of the present invention. The metal may be steel, titanium, a metal alloy or the like. The spine implant 10 is thus representative of various types of medical devices.

The 1-L static spine plate 10 is formed of a unitary body 12 having a center graft window 14 and first and second (or upper and lower) pairs of bone screw bores 16 on opposite longitudinal ends of the body 12. Each bone screw bore 16 is configured to receive a bone screw. Each end pair of bone screw bores 16 allows attachment of respective ends of the spine plate 10 to adjacent vertebra. It should be appreciated that other configurations of one-level static spine plates are contemplated and are intended to be represented by the 1-L static spine plate 10. In accordance with the present principles, the 1-L static spine plate 10 is fabricated from a work hardened metal. The various procedures of the present work hardening technique as described below endue the metal with more strength and flexibility in compression and bending. With the present spine plate, this would be along the curvature and/or a plane normal to curvature of the plate.

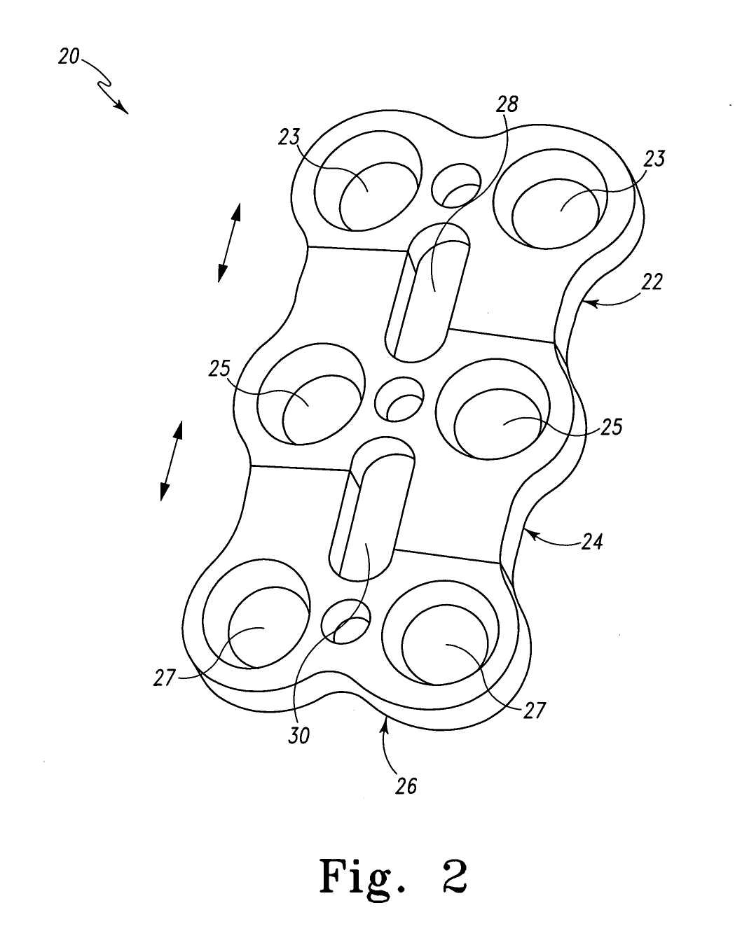

FIG. 2 shows a typical configuration of a medical device being a two-level (2-L) dynamic spine plate generally designated 20 of which provides an example of a configuration of a spinal implant such as may be fabricated from a work hardened metal in accordance with the present method. The metal may be steel, titanium, composite material, an alloy and/or the like. The spine implant 20 is also representative of various types of implants.

The 2-L dynamic spine plate 20 is formed of a first end body 22, a middle body 24 and a second end body 26. The first end body 22 is received on a first end of the middle body 24 while the second end body 26 is received on a second end of the middle body 24 opposite to the first end. As represented by the double-headed arrows, the first end body 22 and the middle body 24 are able to move relative to one another while the second end body 26 and the middle body 24 are able to move relative to one another.

The first end body 22 has a pair of bone screw bores 23 on an end thereof. Each bone screw bore 23 is configured to receive a bone screw. The pair of bone screw bores 23 allows attachment of the first end body 22 to a first vertebra. Configured extensions or legs extend from the bone screw bores 23 that engage configured extensions or legs that extend from one end of the bone screw bores 25 of the middle body 24. In like manner, the second end body 26 has a pair of bone screw bores 27 on an end thereof. Each bone screw bore 27 is configured to receive a bone screw. The pair of bone screw bores 27 allows attachment of the second end body 26 to a second vertebra. Configured extensions or legs extend from the bone screw bores 27 that engage configured extensions or legs that extend from another end of the bone screw bores 25 of the middle body 24. The first and second end bodies 22 and 26 are preferably (and shown as), but not necessarily, identical such that one configuration/piece may be used for both the first and second end bodies 22 and 26.

The middle body 24 and the first end body 22 form a first graft window 28 that is situated between the bone screw bores 25 of the middle body 24 and the bone screw bores 23 of the first end body 22. In like manner, the middle body 24 and the second end body 26 form a second graft window 30 that is situated between the bone screw bores 25 of the middle body 24 and the bone screw bores 27 of the second end body 26. The middle bone screw bores 25 allows attachment of the middle body 24 to a middle vertebra that is between the first and second vertebrae.

It should be appreciated that other configurations of two-level and multi-level dynamic static spine plates are contemplated and are intended to be represented by the 2-L dynamic spine plate 20. In accordance with the present principles, the 2-L dynamic spine plate 20 is fabricated from a work hardened metal in accordance with the present method. The metal may be steel, titanium, an alloy and/or the like. The spine implant 20 is also representative of various types of implants that may be made with the present work-hardened metal.

In accordance with an aspect of the present invention, there is provided a method of producing metal stock suitable for the fabrication or construction of an implant. The metal implant stock comprises a work hardened metal exhibiting an elongated grain structure. Implants such as described herein and others are producible/produced using work hardened metal made in accordance with the present principles. The work hardened metal begins by selecting a work hardenable, bio-compatible metal (work hardenable metal). This may be a steel, titanium, alloy thereof, or other metal or alloy. The work hardenable metal is subjected to a form of fatigue such as by hammering, bending, rolling or the like (forging). The forged metal then undergoes cold or hot rolling (rolling) and annealing. Annealing heat treats the work hardened metal to reorganize its grain structure and return the metal to a softer, more workable state (restores malleability to the metal). These metalworking processes gives the metal implant stock more strength and flexibility in compression and bending. Particularly, these metalworking processes provide a change to the microstructure of the metal. More particularly, the metal microstructure is changed to provide an organized, elongated grain in one direction along the work hardened metal (metal grain elongation).

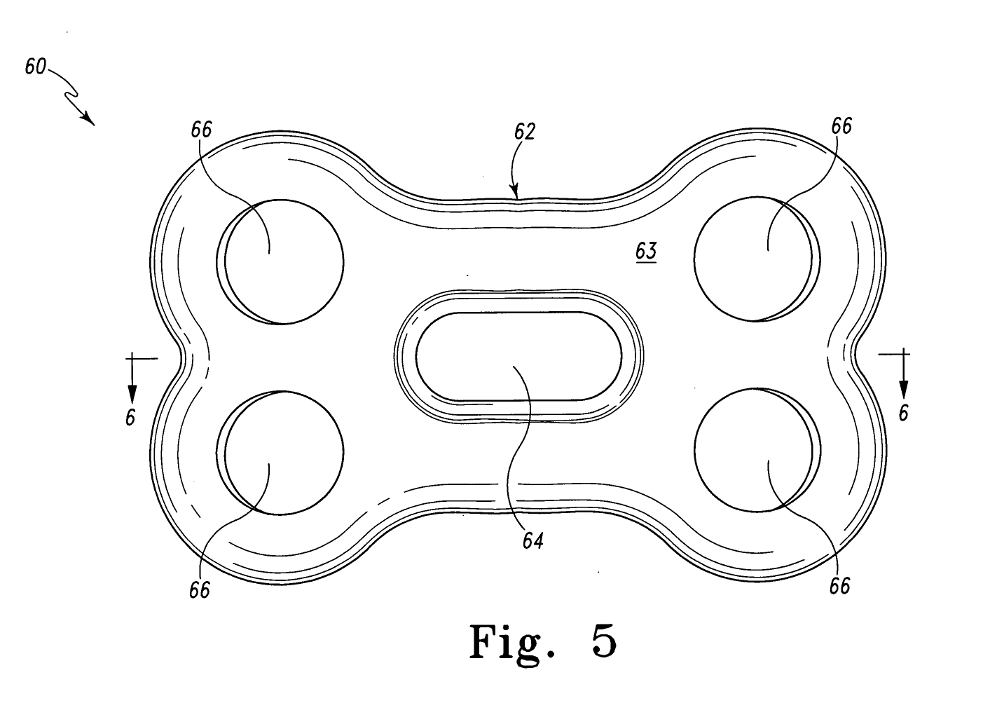

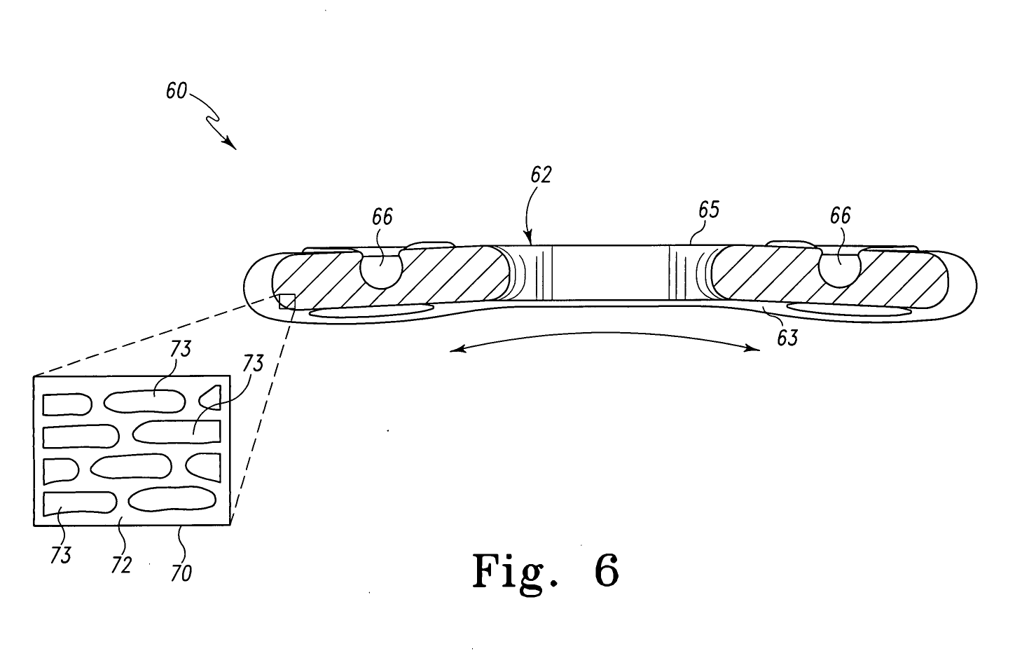

An implant is fabricated from the work-hardened (elongated grain) metal in a manner so as to take advantage of elongated grain properties. As described in connection with FIGS. 5 and 6, an implant 60 is fabricated such that the elongated grains run in the direction of curvature of the implant and/or perpendicular to a plane of stress of the implant. The various procedures of the present work hardening technique endue the metal with more strength and flexibility in compression and bending. It should be appreciated that the work-hardened metal may be work-hardened in other manner so as to produce, instill or modify a particular property of the metal that will be used in the design and/or fabrication of implants. With the present spine plate, this would be along the curvature and/or a plane normal to curvature of the plate.

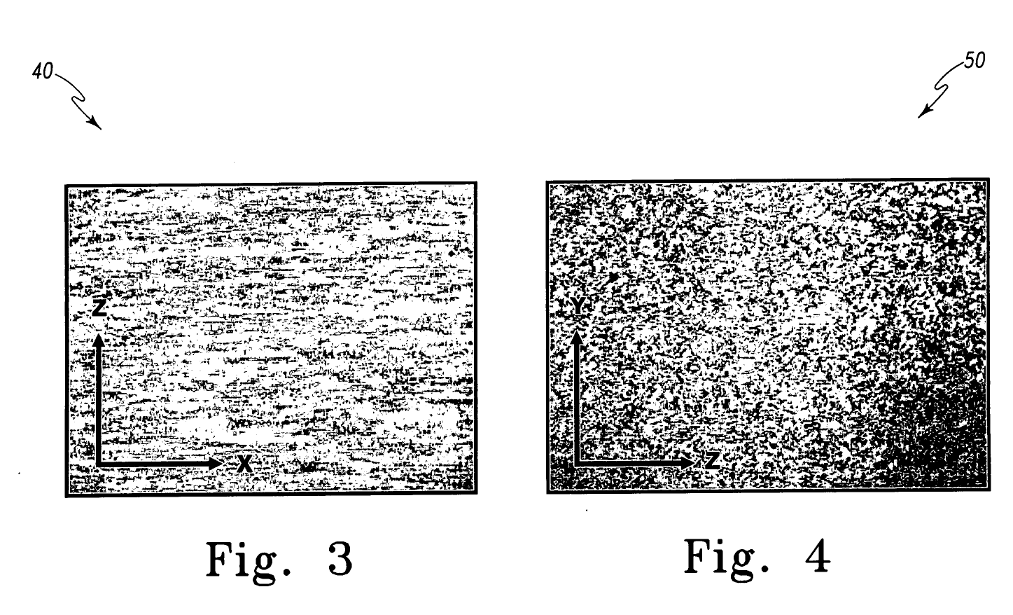

FIG. 3 depicts a sketch of a micrograph of a section of a portion of the present work-hardened metal implant stock and particularly showing an X-Z cross section thereof. It can be seen that the grains are elongated in the X direction as created through the present work hardening method.

FIG. 4 depicts a sketch of a micrograph a section of a portion of the present work-hardened metal implant stock and particularly showing a Y-Z cross section thereof. It can be seen that the grains have no elongation in the Y direction (equlaxed grains).

Referring to FIGS. 5 and 6, there is depicted an implant 60 fashioned with the work-hardened metal such as shown in the micrographs of FIGS. 3 and 4. The implant 60 is a spine plate such as an anterior cervical plate. The implant 60 is defined by a unitary body 62 having a center graft window 64 and first and second (or upper and lower) pairs of bone screw bores 66 on opposite longitudinal ends of the body 62. Each bone screw bore 66 is configured to receive a bone screw. Each end pair of bone screw bores 66 allows attachment of respective ends of the implant 60 to adjacent vertebra.

As illustrated in the enlarged section 70 of the spine plate 60, is can be seen that the spine plate 60 is machined or fabricated from a work hardened metal 72 whose grain structure 73 has been elongated. Particularly, it can be seen that the elongated grain structure of the work-hardened metal is in a plane normal to the curvature (represented by the double-headed arrow) of the spine plate. The present work hardening technique as described herein endues the implant metal with more strength and flexibility in compression and bending along the elongated grain structure. This characteristic (and others) may also be exploited in other medical devices.

Other medical devices such as implants and implant configurations may be fabricated from the present work-hardened metal to exploit its elongated grain structure. As well, other medical devices such as instruments may take advantage of an elongated grain structure. For instance, the implant fabricated from a work-hardened metal may be machined in a certain orientation such that the elongated grains of the work-hardened metal align with a particular axis of the implant. In general, in accordance with an aspect of the present invention, material selection and material property change and/or exploitation of a metal are utilized to construct implants.

Moreover, other work-hardened metal properties may be utilized for fabrication of a medical device wherein the metal properties give the medical device characteristics that aid in the performance of the medical device.

While the invention has been illustrated and described in detail in the drawings and foregoing description, the same is to be considered as illustrative and not restrictive in character, it being understood that only preferred embodiments have been shown and described and that all changes and modifications that come within the spirit of the invention are desired to be protected.

What is claimed is:

1. A method of fabricating a medical device comprising the steps of:
work hardening a work-hardenable metal to achieve a desired microstructure of the metal; and
producing a medical device in accordance with the achieved microstructure of the metal.

2. The method of claim 1, wherein the step of work hardening a work-hardenable metal comprises work hardening by one of forging, cold rolling, hot rolling, and annealing.

3. The method of claim 1, wherein the step of work hardening a work-hardenable metal comprises work hardening by one or more of forging, cold rolling, hot rolling, and annealing.

4. The method of claim 1, wherein the step of work hardening a work-hardenable metal comprises work hardening by a series of forging, cold rolling, hot rolling, and annealing.

5. The method of claim 1, wherein the achieved microstructure of the metal comprises grain elongation.

6. The method of claim 5, wherein the step of producing a medical device with the achieved microstructure of the metal includes the step of orienting the medical device relative to the plane of grain elongation.

7. The method of claim 6, wherein the medical device comprises a spine plate.

8. The method of claim 7, wherein the elongated grains of the work hardened metal are oriented in a plane normal to a longitudinal axis of the spine plate.

9. The method of claim 6, wherein the medical device comprises a curved spine plate with the elongated grains of the work hardened metal oriented in a plane normal to the curvature of the spine plate.

10. A medical device comprising:
a body fabricated from a work-hardenable metal that has been work hardened to achieve a desired microstructure of the metal; and
wherein the achieved microstructure has characteristics that aid in performance of the medical device.

11. The medical device of claim 10, wherein the work-hardened metal is achieved by one of forging, cold rolling, hot rolling, and annealing.

12. The medical device of claim 10, wherein the of work hardening a work-hardenable metal comprises work hardening by one or more of forging, cold rolling, hot rolling, and annealing.

13. The medical device of claim 10, wherein the step of work hardening a work-hardenable metal comprises work hardening by a series of forging, cold rolling, hot rolling, and annealing.

14. The medical device of claim 10, wherein the achieved microstructure of the metal comprises grain elongation.

15. The medical device of claim 14, wherein the body is fabricated through orienting the medical device relative to the plane of grain elongation.

16. The medical device of claim 15, wherein the medical device comprises a spine plate.

17. The medical device of claim 16, wherein the elongated grains of the work hardened metal are oriented in a plane normal to a longitudinal axis of the spine plate.

18. The medical device of claim 15, wherein the medical device comprises a curved spine plate with the elongated grains of the work hardened metal oriented in a plane normal to the curvature of the spine plate.
